(19) United States
(12) Patent Application Publication
(10) Pub. No.: US 2004/0191775 A1
(43) Pub. Date: Sep. 30, 2004

(54) Methods of analyzing genes affected by caloric restriction or caloric restriction mimetics

(76) Inventors: Stephen R. Spindler, Riverside, CA (US); Joseph M. Dhabi, Riverside, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/387,786

(22) Filed: Mar. 12, 2003

(51) Int. Cl. C12Q 1/68

(52) U.S. Cl. 435/6

(57) ABSTRACT

A method of analyzing genes. In one embodiment a method of analyzing genes comprises administering a first type of CR dietary program for a first period of time for a first sample; administering a second dietary program for the first sample after the first period of time; and administering a control diet to a second sample. The gene expression effects between the first sample and the second sample are analyzed.

### BACKGROUND

1. Field

Many aspects of this disclosure relate to methods of analyzing genes affected by caloric restriction (CR) or CR mimetics. For example, methods of identifying genes and categorizing genes that are affected, altered, expressed, down regulated, or otherwise changed by CR or CR mimetics are disclosed.

2. Discussion of Related Art

A major goal of pharmaceutical research has been to discover ways to reduce morbidity and delay mortality. Several decades ago it was discovered that a decrease in caloric intake, termed caloric restriction (CR) can significantly and persistently extend healthy life in animals; see for example, Weindruch, et. al., The Retardation of Aging and Disease by Dietary Restriction, (Charles C. Thomas, Springfield, Ill.), 1988. CR remains the only reliable intervention capable of consistently extending lifespan and reducing the incidence and severity of many age-related diseases, including cancer, diabetes and cardiovascular disease. Additionally, physiological biomarkers linked to lifespan extension in rodents (e.g., mice, rabbits, shrews, and squirrels) and monkeys that have been subjected to CR have been shown to be associated with enhanced lifespan in humans; see for examples, Weyer, et. al., Energy metabolism after 2 years of energy restriction: the biosphere 2 experiment, Am. J. Clin. Nutr. 72, 946-953, 2000, and Roth, et. al., Biomarkers of caloric restriction may predict longevity in humans, Science 297, 811, 2002. A study by Walford et. al. indicated that healthy nonobese humans on CR diets show physiologic, hematologic, hormonal, and biochemical changes resembling those of rodents and monkeys on such CR diets. See Walford, et. al., Calorie Restriction in Biosphere 2: Alternations in Physiologic, Hematologic, Hormonal, and Biochemical Parameters in Humans Restricted for a 2-Year Period, J. Gerontol: Biol. Sci. 57A, 211-224, 2002. These preliminary findings suggest that the anti-aging effects of CR may be universal among all species. The molecular-genetic processes that lead to lifespan extension in animals may extend lifespan in humans. CR thus brings many benefits to animals and humans.

It has been known that CR affects gene expression. Understanding what kind of genes or what groups of genes CR affects will be advantageous in the field of genomic medicine. The understanding of the dynamics of the changes in gene expression in response to CR has been a daunting task. There is currently no method that allows the understanding of the relatedness of genes and how certain genes are affected by similar CR treatments. Understanding of the dynamics of the changes in gene expression in response to CR is important and can lead to more understanding of the behavior, structure, and function of genes. Understanding the behavior, structure, and function of genes also enables grouping of genes that behave similarly and discovering ways to regulate genes as a group. Motif discovery involves taking co-regulated genes and deducing the signal transduction systems that are affected by CR and these systems can be targets for interventions (e.g., drug therapies).

### SUMMARY OF DISCLOSURE

In one embodiment, a method of analyzing genes comprises administering a first type of a CR dietary program for a first period of time for a first sample; administering a second dietary program for the first sample after the first period of time; and, administering a control diet to a second sample. The gene expression effects or other effects between the first sample and the second sample are analyzed.

In another embodiment, a method for identifying targets for interventions comprises comparing gene expression levels or protein activity levels in a sample exposed to a first type of CR and to a second type of CR. Genes that appear to have similarity in the responses of both the first and the second types of CR are identified.

In another embodiment, a method for identifying a compound that potentially reduces collagen accumulation in myocardium comprises obtaining control data from an administering of a CR dietary program to one group and administering a dosage of a compound to another group. At least one collagen measurement resulting from the CR dietary program is compared to at least one collagen measurement resulting from the administering a dosage of the compound. The compound is identified to be potentially effective in reducing collagen accumulation based at least in part on the comparison between the collagen measurement resulting from the CR dietary program and the collagen measurement resulting from the administering of the compound.

In another embodiment, a method for identifying a compound that potentially reduces collagen accumulation in myocardium and blood vessels comprises obtaining control data from an administering of a CR dietary program to a first mammalian group. The CR dietary program includes at least one of a long-term CR (LT-CR) dietary program and a short-term CR (ST-CR) dietary program. The method also comprises administering an effective dosage of a compound to a second mammalian group. At least one of collagen gene expression or collagen accumulation between the first mammalian group and the second mammalian group are compared. The compound is chosen to be potentially effective in reducing collagen accumulation based at least in part on comparing the collagen gene expression or collagen accumulation between the first mammalian group and the second mammalian group.

A method of fractionating genetic information into groups is also disclosed. Control data from an administering of a long-term control (LT-CON) dietary program is obtained. A first sample group is subjected to a LT-CON dietary program for a first predetermined period after which, the first sample group is switched from the LT-CON dietary program to a ST-CR dietary program for a second predetermined period. A second sample group is subjected to a LT-CR dietary program for the first predetermined period after which, the second sample group is divided to a third sample group that is switched to a short-term control (ST-CON) dietary program and a fourth sample group that is maintained on the same LT-CR dietary program for the second predetermined period. The effects among the first sample group, the third sample group, and the fourth sample group are compared to the control data and to each other.

These and other features and advantages of embodiments of the present invention will be more readily apparent from the detailed description of the embodiments, set forth below, taken in conjunction with the accompanying drawings.

### BRIEF DESCRIPTION OF THE DRAWINGS

The present invention is illustrated by way of example and not limitation in the figures of the accompanying drawings, in which like references indicate similar elements and in which:

FIG. 1 illustrates an exemplary dietary regimen scheme that various groups of samples are subjected to;

FIGS. 2A-2B illustrate how genes are categorized into clusters based on various caloric restriction dietary regimens;

FIG. 3 illustrates exemplary results of real time RT-PCR (reverse transcriptase-PCR) data validating microarray data to confirm gene changes; (PCR is Polymerase Chain Reaction)

Table 1 illustrates exemplary primer sequences for real time RT-PCR that can be used for some embodiments of the present invention; and

Table 2 illustrates some effects of LT-CR, ST-CR and ST-CON dietary regimens.

The features of the described embodiments are specifically set forth in the appended claims. However, the embodiments are best understood by referring to the following description and accompanying drawings, in which similar parts are identified by like reference numerals.

### DETAILED DESCRIPTION

In the following description, for purposes of explanation, numerous specific details are set forth in order to provide a thorough understanding of the exemplary embodiments of the present invention. It will be evident, however, to one skilled in the art, that these embodiments may be practiced without these specific details. In other instances, specific structures and methods have not been described so as not to obscure the present invention. The following description and drawings are illustrative of the invention and are not to be construed as limiting the invention.

Throughout the discussion, the following terminologies are used. A control (CON) dietary program or regimen refers to normal feeding programs (e.g., 93 kcal/week for mice test group). A CR dietary program refers to feeding program with a reduced amount of calories (e.g., 77 kcal/week or 52 kcal/week for mice test group). It is to be appreciated that the number of calories per week can be modified to adjust to what is considered normal for a particular test group. A LT-CR dietary program refers to a reduced dietary regimen for a long duration of time, e.g., for more than 8 weeks in the case of mice or between about several months to about 36 months or to about the end of life in some cases. A ST-CR dietary program refers to a reduced dietary regimen for a short duration of time, e.g., for about 8 weeks or less than 8 weeks in the case of mice or between about several months to about 36 months in some cases. In certain situations, a dietary program may be a ST-CR dietary program which runs until about the end of life when a ST-CR dietary program is begun after a control dietary program (e.g., the control dietary program was administered to one or more animals in a test group for a long duration and the dietary program for these animals was switched to a ST-CR dietary program for the rest of the animals' lives. It is to be appreciated that the number of weeks that constitute short or long duration of time for a dietary program or regimen can be varied depending on experimental designs, test groups, mammalian species, etc.

In addition, a ST-CR group refers to a test group or a sample group that is subjected to a ST-CR dietary regimen. A ST-CON group refers to a test group or a sample group that is subjected to a control dietary regimen for a short duration of time relative to another dietary regimen for a longer period of time. A LT-CR group refers to a test group or a sample group that is subjected to a LT-CR dietary regimen. A LT-CON refers to a test group or a sample group that is subjected to a control dietary regimen for a long duration of time.

Moreover, a long-term drug group refers to a test group or a sample group that is subjected to a dietary regimen that includes administration of at least one compound, test compound or a pharmaceutical agent for a long duration of time, wherein the compound can be a CR mimetic candidate or a potential CR mimetic candidate. A short-term drug group refers to a test group or a sample group that is subjected to a dietary regimen that includes administration of at least one compound, test compound, or a pharmaceutical agent for a short duration of time, wherein the compound can be a CR mimetic candidate or a potential CR mimetic candidate. A short-term drug withdrawn group refers to a test group or a sample group that is subjected to a withdrawal of the compound that is administered to the group as described in either the long-term drug group or the short-term drug group where the withdrawal is for a short term.

Exemplary embodiments are described with reference to specific configurations and techniques. The exemplary embodiments pertain to methods of analyzing effects induced by CR or CR mimetics. The effects include at least one of changes in gene expression levels (e.g., mRNA levels), changes in protein levels, changes in protein activity levels, changes in carbohydrate or lipid levels, changes in nucleic acid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function. The following discussion focuses on several exemplary methods of identifying and categorizing genes that are expressed, not expressed, or otherwise altered (e.g., negatively or positively regulated) as induced by CR or CR mimetics. CR mimetic refers to a compound, a test compound, an agent, a pharmaceutical agent, or the like, that reproduces at least some effects induced by CR. It is to be appreciated by one skilled in the art that the exemplary methods are not limited only to analyzing genes expressions that are affected by CR or CR mimetics but are also to include changes in physiological biomarkers such as changes in protein, changes in protein activity, changes in levels of nucleic acids, changes in carbohydrate levels, changes in lipid levels, changes in rate of protein or nucleic acid synthesis, changes in protein or nucleic acid stability, changes in protein or nucleic acid accumulation levels, changes in protein or nucleic acid degradation rate, and changes in protein or nucleic acid structure or function, and the like.

Currently, CR when started either early in life or in middle age, represents the best established paradigm of aging retardation in mammals. See for example, Weindruch, et. al., The Retardation of Aging and Disease by Dietary Restriction, (C. C. Thomas, Springfield, Ill., 1988). The effects of CR on age-related parameters are broad. CR increases maximum lifespan, reduces and delays the onset of age related diseases, reduces and delays spontaneous and induced carcinogenesis, suppresses autoimmunity associated with aging, and reduces the incidence of several age-induced diseases (Weindruch, supra, 1988).

Even though CR brings many beneficial effects to animals and humans, it is not likely that many will avail themselves of a CR lifestyle. As is known, it is difficult for any animal or human to maintain a dietary program. There is thus a need to identify, evaluate, and develop compounds and/or drugs and/or mechanisms that are capable of mimicking the beneficial effects of CR without the reduction of dietary calorie intake as required by CR dietary programs. Additionally, CR or CR mimetics may affect some genes in similar manners. Understanding of the dynamics of the changes in gene expression in response to CR or CR mimetics is important since it may allow for more understanding into the behavior, structure, and function of genes in a particular group. Furthermore, understanding the behavior, structure, and function of genes, how they associate with each other as a group, and how they respond to CR or CR mimetics enables creating ways to regulate genes as a group. There is thus a need to identify the dynamics of the changes in gene expression for groups of genes and to identify the relatedness of genes to one another based on similar treatments. When the dynamics of the changes in gene expression for groups of genes are better understood, it becomes easier and more efficient to regulate genes as a group or groups using fewer compounds and mechanisms.

In one embodiment, a mammalian sample group is chosen. The sample group can be rodents such as laboratory mice. The mice are divided into groups, each of which will undergo a different treatment. One group of mice is subjected to a CR dietary program (reduced diet) generating a CR group. Another group of mice can be a control group, which is subjected to a control (normal) dietary program generating a control group. The CR group is then divided into two sub-groups, one of which is switched to the control dietary program while the other is maintained on the same CR dietary program. The control group is also divided into two sub-groups, one of which is switched to a CR dietary program while the other is maintained on the same control dietary program. Under these switching of dietary regimens, genes that are similarly affected by a certain CR regimen individually and as a group can also be determined. As will be apparent below, switching the dietary regimen affects certain genes or groups of genes in the same way. This allows for the discovery of regulatory factors and signal transduction pathways that control gene expression. In another embodiment, a compound (or a CR mimetic) can also be administered to a group of mice in similar manner, for example, switching a control diet group to a test compound group. From the results, it can be determined whether the compound can reproduce or mimic at least some effects that are caused by CR. It will be recognized that the various embodiments described herein can be used with non-mammal organisms such as insects, nematodes, yeast, bacteria, and other organisms. In some situations, techniques may be performed in these non-mammal organisms and then candidate drugs, discovered in those organisms, can be tested in mammals (e.g., humans).

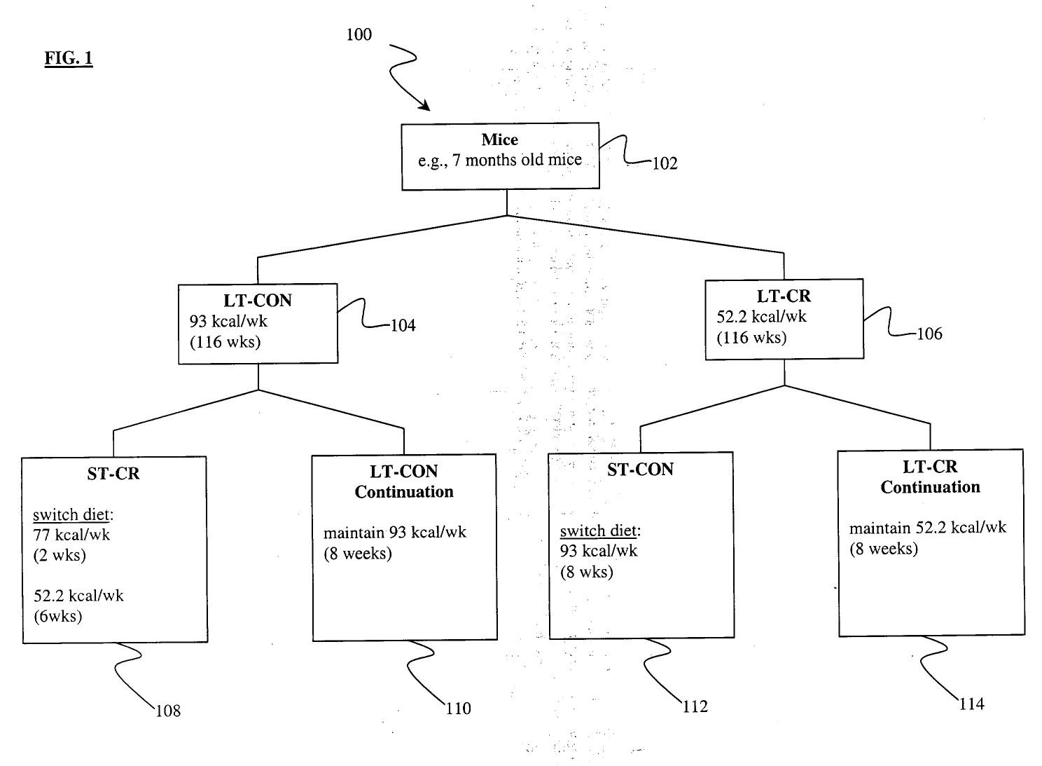

FIG. 1 illustrates an exemplary scheme 100 of the various dietary regimens for mammalian samples. In one embodiment, the mammalian samples are mice. Male mice of the long-lived F1 hybrid strain B6C3F1 were fed and maintained as described in Dhahbi, et., al., Caloric intake alters the efficiency of catalase mRNA translation in the liver of old female mice, J.Gerontol.A Biol.Sci.Med.Sci.; 53: B180-B185, 198, which is hereby incorporated by reference. Briefly, the mice were purchased from Jackson Laboratories (Bar Harbor, Me. 04609). For the first seven months, mice were fed rodent diet No. 5001 (TMI Nutritional International LLC, Brentwood, Mo. 63044). At seven months, all mice were individually housed. The seven-month old mice are indicated as mice group 102 as shown in FIG. 1. The mice from the group 102 were randomly assigned to one of two groups, a LT-CON group 104 and a LT-CR group 106. Each mouse in the LT-CON group 104 was subjected to a LT-CON dietary program with feeding of 93 kcal per week of a semi-purified control diet in 1 gm pellets (AIN-93M, Diet No. F05312, BIO-SERV, Frenchtown, N.J., 08825). A complete list of diet ingredients can be found on the Harland Teklad website http://www.teklad.com/custom/index.htm. Each mouse in the LT-CR group 106 was subjected to a LT-CR dietary program with feeding of 52.2 kcal per week of a semi-purified CR diet (AIN-93M 40% Restricted, Diet No. F05314, BIO-SERV).

In one embodiment, after 29 months of age (116 weeks), the mice from both the LT-CON group 104 and the LT-CR group 106 were subjected to a crossover (or switching) experiment in which LT-CR and LT-CON mice were switched to the opposite dietary regimen for 2 months (8 weeks). In one embodiment, half of the mice from the LT-CON group 104 were switched to a ST-CR dietary program for 8 weeks generating a ST-CR group 108. The other half of the mice from the LT-CON group 104 continued with the LT-CON dietary program for 8 weeks generating a LT-CON continuation group 110. Note that there is no change in the dietary regimen for the mice that are not switched to the ST-CR dietary program. Hence, for clarity of discussion, the group of mice that is maintained on the LT-CON dietary program is referred to as a LT-CON continuation group. Thus, a LT-CON continuation group may simply refer to a group of mice that is subjected to a LT-CON dietary program. Additionally, half of the mice from the LT-CR group 106 were switched to a short-term ST-CON dietary program for 8 weeks generating a ST-CON group 112. The other half of the mice from the LT-CR group 106 continued with the LT-CR dietary program for 8 weeks generating a LT-CR continuation group 114. There is no change in the dietary regimen for the mice that are not switched to the ST-CON dietary program. The group of mice that are continued with the LT-CR dietary program is thus referred to as a LT-CR continuation group, which simply refers to a group of mice that is subjected to a LT-CR dietary program.

In one embodiment, the mice from the ST-CR group 108 were mice from the LT-CON group 104 that were switched from a 93 kcal per week diet to a 77 kcal per week diet for 2 weeks, followed by a 52.2 kcal per week diet for 6 weeks. The mice from the ST-CON group 112 were the mice from the group LT-CR 106 that were switched to a control dietary program for 8 weeks in which the mice were switched from a 52.2 kcal per week diet to a 93 kcal per week diet. Thus, in one embodiment, the switching of the groups of mice to different dietary programs generates 4 sample groups, LT-CON continuation group 110, LT-CR continuation group 114, ST-CON group 112, and ST-CR group 108. In one embodiment, each group includes 4 mice.

All mice were killed at 124-weeks of age (31 months). Mice from all groups were fasted for 48 hours before killing. Mice were killed by cervical dislocation, and hearts rapidly excised, rinsed in PBS to remove blood, and flash frozen in liquid nitrogen. No signs of pathology were detected in any of the animals used. All animal use protocols were approved by an institutional animal use committee.

It is also to be noted that control data can be obtained from a prior study, the results of which are recorded as opposed to a control group of mice subjected to a control diet program concurrently with the test groups of mice as illustrated in FIG. 1. Thus, the control data may be obtained from an administering of a control diet program which was previously performed. This control data may be obtained once and stored for recall in later screening studies for comparison against the results in the later screening studies. Similarly, gene expression levels from LT-CR or ST-CR (or other types of measurements such as protein levels, nucleic acid levels, carbohydrate levels, lipid levels) may be evaluated and recorded once for recall in later screening studies for comparison against the results in the later screening studies. Of course, it is typically desirable to have the prior stored studies have a similar (if not identical) set of genes (or other parameters such as proteins) relative to the genes (or other parameters) in the later screening studies in order to perform a comparison against a similar set of genes or other parameters.

The effects caused to each of the four groups of mice (LT-CON continuation group 110, LT-CR continuation group 114, ST-CON group 112, and ST-CR group 108) were compared to each other. In one embodiment, the effects were used to determine the effects of CR on gene expression caused by each of the different dietary programs. In one embodiment, the effects of LT-CR on gene expression were determined by comparing the results between the LT-CON continuation group 110 and the LT-CR continuation group 114. The effects of ST-CR were determined by comparing the results between the LT-CON continuation group 110 and the ST-CR group 108. The effects of ST-CON were determined by comparing the results between the LT-CON continuation group 110 and the ST-CON group 112.

In other embodiments, a test compound (or test compounds) that is a CR mimetic candidate or a potential CR mimetic can be administered to the a group of mice. For example, in addition to, or instead of, switching some of the LT-CON group 104 to the ST-CR dietary program (e.g., to generate the ST-CR group 108), some of the mice from the LT-CON group 103 can be switched to a dietary program that includes the test compound. The effects of this test compound can then be determined by comparing the results between the LT-CON group and the test compound group in the same way that the results for the ST-CR is obtained by comparing the results between the ST-CR group 108 and the LT-CON continuation group 110. Similarly, a group of mice can be subjected to a dietary program that includes the test compound for the same duration as the LT-CR dietary program generating for example, a long-term drug group. After this duration, some of the mice from this group are subjected to a control dietary regimen without the test compound generating a short-term drug withdrawal group. One effect that can be determined from comparing the long-term drug group and the short-term drug withdrawal group may include determining whether the effects of the test compound are reversible by a control dietary regimen or by withdrawing the test compound.

In one embodiment, specific mRNA levels from the hearts of mice from all of the various test groups were measured. It is to be appreciated that measuring specific mRNA levels is only one exemplary method of identifying the effects caused by various dietary regimens or test compounds. Other methods such as those conventionally used for measuring specific protein activity levels, specific protein level changes, specific carbohydrate level changes, specific lipid level changes, and specific nucleic acid levels can be used. Other heart RNA was isolated from frozen tissue fragments by homogenization in TRI Reagent (Molecular Research Center, Inc., Cincinnati, Ohio) with a Tekmar Tissuemizer (Tekmar Co., Cincinnati, Ohio) as described by the suppliers. mRNA levels were measured using the Affymetrix U74v2A high-density oligonucleotide arrays according to the standard Affymetrix protocol (Affymetrix, Santa Clara, Calif.). Briefly, cDNA was prepared from total RNA from each animal using Superscript Choice System with a primer containing oligo(dT) and the T7 RNA polymerase promoter sequence. Biotinylated cRNA was synthesized from purified cDNA using the Enzo BioArray High Yield RNA Transcript Labeling Kit (Enzo Biochem). cRNA was purified using RNeasy mini columns (Qiagen, Chatsworth, Calif.). An equal amount of cRNA from each animal was separately hybridized to U74v2A high-density oligonucleotide arrays. The arrays were hybridized for 16 hours at 45° C. After hybridization, arrays were washed, stained with streptavidin-phycoerythrin, and scanned using a Hewlett-Packard GeneArray Scanner. In one embodiment, image analysis and data quantification were performed using the Affymetrix GeneChip analysis suite v5.0.

In embodiments where the Affymetrix Gene Chip analysis suite are used, the U74vA array contains targets for more than 12,422 mouse genes and expressed sequence tags (ESTs). Each gene or EST is represented on the array by 20 perfectly matched (PM) oligonucleotides and 20 mismatched (MM) control probes that contain a single central-base mismatch. All arrays were scaled to a target intensity of 2500. The signal intensities of PM and MM were used to calculate a discrimination score, R, which is equal to (PM−MM)/(PM+MM). A detection algorithm utilized R to generate a detection p-value and assign a Present, Marginal or Absent call using Wilcoxon's signed rank test. Details of this method can be found in Wilcoxon F. Individual Comparisons by Ranking Methods, Biometrics 1, 80-83, 1945, and Affymetrix, I. New Statistical Algorithms for Monitoring Gene Expression on GeneChip Probe Arrays, Technical Notes 1, Part No. 701097 Rev. 1, 2001. Only genes that were “present” in at least 2 out of 4 arrays per experimental group were considered for further analysis. In addition, genes with signal intensity lower than the median array signal intensity in any of the 16 arrays were eliminated from the analysis. These selection criteria reduced the raw data from 12,422 genes to only 3456 genes which were considered for further analysis.

In one embodiment, to identify differentially expressed genes between any two groups, each of the 4 samples in one group was compared with each of the 4 samples in the other group, resulting in 16 pairwise comparisons. These data were analyzed statistically using a method based on Wilcoxon's signed rank test. Difference values (PM-MM) between any two groups of arrays were used to generate a one-sided p-value for each set of probes. Default boundaries between significant and not significant p-values were used. (See Affymetrix, I. New Statistical Algorithms for Monitoring Gene Expression on GeneChip Probe Arrays, mentioned above, for more details). In one embodiment, genes are considered to have changed expression if the number of increase or decrease calls was 8 or more of the 16 pairwise comparisons, and an average fold change, derived from all 16 possible pairwise comparisons, was 1.5-fold or greater. Empirically, these criteria for identifying gene expression changes can be reliably verified by methods such as Western blot, Northern blot, dot blot, primary extension, activity assays, real time PCR, and real time RT-PCR (reverse transcriptase. PCR). Gene names were obtained from the Jackson Laboratory Mouse Genome Informatics database as of Aug. 1, 2002.

In one embodiment, the effects caused by LT-CR, ST-CR, and ST-CON dietary regimens are listed in Table 2. These effects are illustrated in terms of fold changes. The numbers in the LT-CR column represent the average fold change in specific mRNA derived from all 16 possible pairwise comparisons among individual mice from the LT-CR and LT-CON groups (n=4). The numbers in the ST-CR column represent the average fold change in specific mRNA derived from all 16 possible pairwise comparisons among individual mice from the ST-CR and LT-CON groups (n=4). The numbers in the ST-CON column represent the average fold change in specific mRNA derived from all 16 possible pairwise comparisons among individual mice from the ST-CON and LT-CON groups (n=4). Where there is no change in gene expression, an “NC” is denoted. In one embodiment, the ratios of the fold changes are determined to illustrate the effects on gene expression. For each ratio, the numerator is the level of expression of each gene from the LT-CR, ST-CR, or ST-CON group, and the denominator is the level of expression of that gene in the LT-CON group. For example, the fold changes in gene expression caused by LT-CR is the ratio of the level of expression of each gene in the LT-CR group divided by the level of expression of that gene in the LT-CON group. The fold changes in gene expression caused by ST-CR is the ratio of the level of expression of each gene in the ST-CR group divided by the level of expression of that gene in the LT-CON group. The fold changes in gene expression caused by ST-CON is the ratio of the level of expression of each gene in the ST-CON group divided by the level of expression of that gene in the LT-CON group.

As mentioned above, gene expressions can be validated by real time RT-PCR. In one embodiment, the expression of a total of 9 genes randomly chosen from among the genes which changed expression was examined by real time RT-PCR using total cardiac RNA purified from the mice used in the microarray studies. Total RNA was treated with DNase I (Ambion Inc., Austin, Tex.) and used to synthesize cDNA in a 20 μl total volume reaction. Briefly, 2 μg of total RNA were incubated with 250 ng random primer (Promega, Madison, Wis.) for 5 min at 75° C., and then on ice for 5 min. 2 μl of 0.1 M DTT, 4 μl of 5× buffer, 4 μl of 2.5 mM dNTP, 100 U (units) reverse transcriptase (Invitrogen, Carlsbad, Calif.), and 16.5 U RNase inhibitor (Promega) were added and incubated for 2 hr at 37° C. The reaction was stopped by boiling for 2 min at 100° C. An identical reaction without the reverse transcriptase was performed to verify the absence of genomic DNA. All samples were reverse-transcribed at the same time and the resulting cDNA was diluted 1:4 in water and stored at −80° C.

Relative quantification with real-time, two-step real time RT-PCR was performed with Quantitect SYBR Green PCR kit (Qiagen, Hilden, Germany) and an ABI PRISM 7700 Sequence Detection System (Applied Biosystems, Foster City, Calif.), according to the manufacturer's instructions. Primers were designed using Netaffx analysis center and verified against the public databases to confirm unique amplification products (http://www.affymetrix.com/analysis/index.affx and http://www.ncbi.nlm.nih.gov), (Table 1). Primers for transcription factor S-II were amplified in parallel with the genes of interest. Transcription factor S-II was used as a reference gene because its mRNA levels are unaffected by a CR diet. For each gene, single real time RT-PCR was performed with each individual mRNA sample obtained from mice from each of the sample groups, for example, the LT-CON continuation group 110 (n=4), the LT-CR continuation group 114 (n=4), the ST-CON group 112 (n=4) and the ST-CR group 108 (n=4). Briefly, real time RT-PCR was carried out in 25 μl volumes containing 2 in of diluted cDNA, 1X SYBR Green PCR Master Mix, 0.5 mM of each forward and reverse primers, and 0.5 unit uracil N-glycosylase. The reactions were incubated for 2 min at 50° C. to allow degradation of contaminating cDNA by uracil N-glycosylase, and 15 min at 95° C. to activate HotStarTaq DNA polymerase. Target amplification reactions were cycled 40 times with denaturation at 94° C. for 15 sec, annealing at 60° C. for 30 sec, and extension at 72° C. with 30 sec. To confirm amplification specificity, the PCR products from each primer pair were subjected to a melting curve analysis and subsequent agarose gel electrophoresis.

The heart tissue from each mouse from each of the test groups including the LT-CON continuation group 110, the LT-CR continuation group 114, the ST-CON group 112, and the ST-CR group 108 was isolated for determination of effects of each of the different treatments. For example, profiles such as gene expression levels, nucleic acid levels, protein levels, protein activity levels, carbohydrate levels, and lipid levels, to name a few, can be analyzed for the hearts isolated from mice from the various groups. The methods for such analysis are well known in the art. Some embodiments of the present invention focus on the determination of changes in gene expression levels. It is to be noted that such determination is not the only method that can be used to analyze the effects of CR, LT-CR, ST-CR, switching of the CR dietary programs, and mimetic compounds.

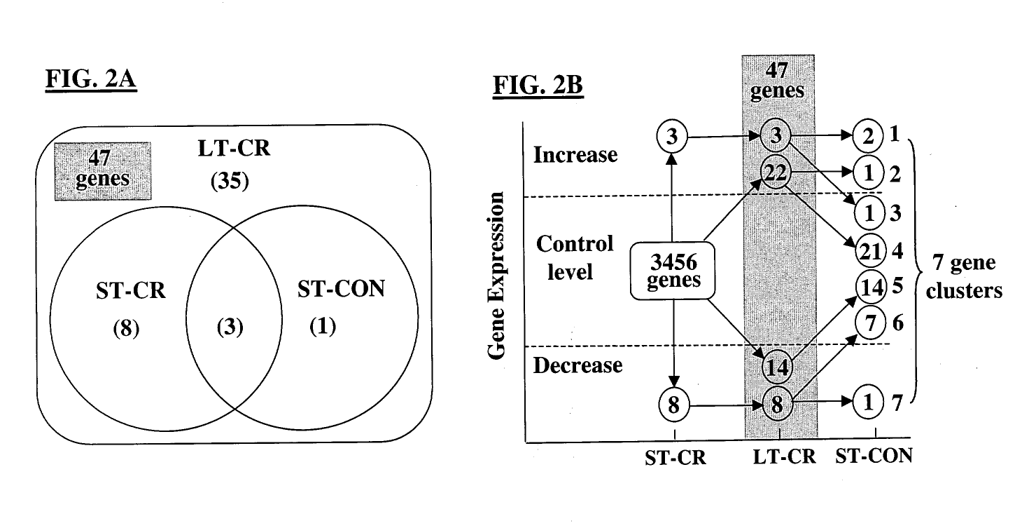

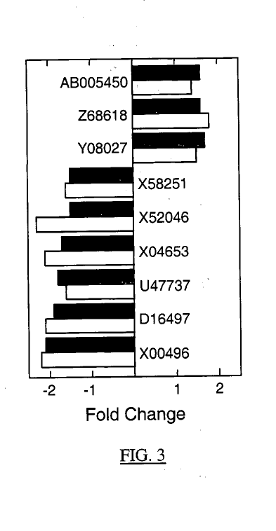

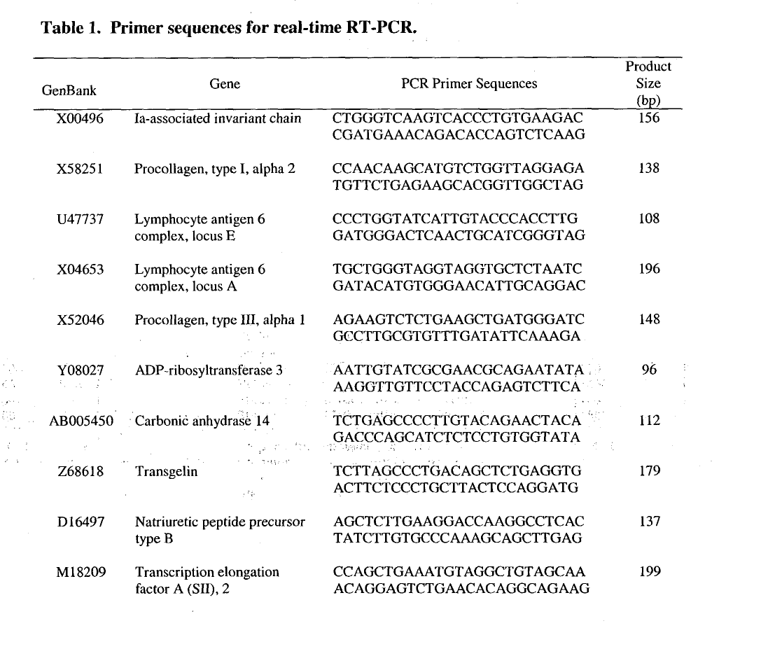

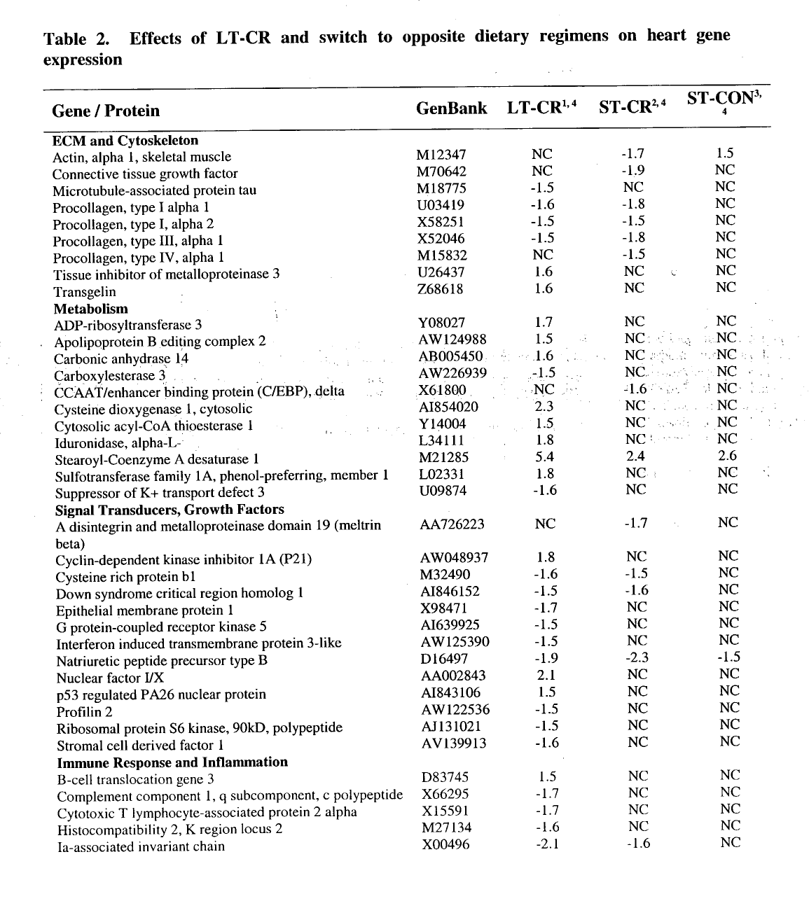

In one embodiment, microarray assessment of the relative levels of mRNA of 12,422 genes and ESTs revealed that 47 genes in the heart changed expression with a LT-CR dietary program as illustrated in FIG. 2A. These differentially expressed genes are further grouped into categories by their putative functions as illustrated in Table 2. LT-CR and ST-CR affected the expression of genes whose products are components of extracellular matrix and cytoskeleton, intermediary metabolism, immune and stress responses and signal transduction.

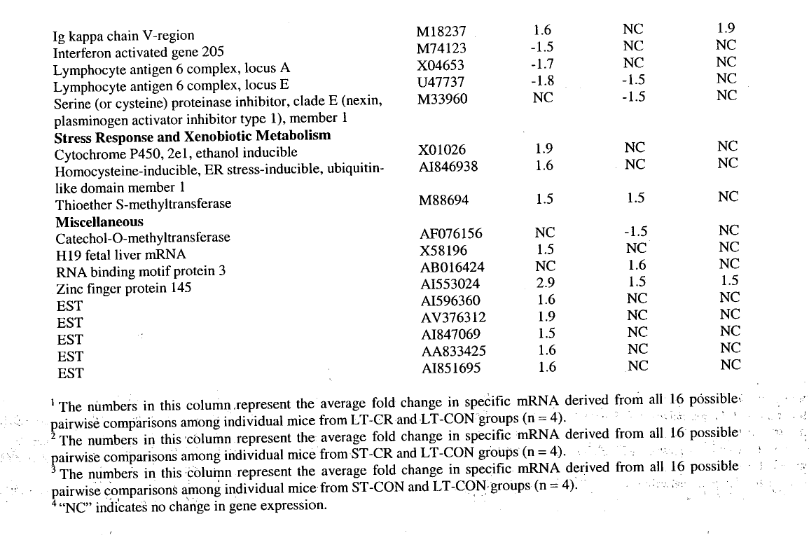

Expression of a subset of the genes listed in Table 2 was also measured using real time RT-PCR. In FIG. 3, 9 randomly chosen genes (with gene names AB005450, Z68618, Y08027, X58251, X52046, X04653, U47737, D16497, and X00496) were monitored by quantitative PCR. As illustrated in FIG. 3, PCR confirmed the changes found by microarray for each of the 9 chosen genes. As can be seen from this figure, the fold changes are in the same direction and are substantially similar in the amount of the fold changes. The results in FIG. 3 indicate that the analytical methods used here reliably identified genes that change expression.

In one embodiment, to elucidate the dynamics of the changes in gene expression in response to caloric intake, LT-CR and LT-CON mice were subjected to an 8-week switch to an opposite diet. For instance, as previously mentioned, some mice from the LT-CR group were switched from the LT-CR dietary program to the ST-CON dietary program (FIG. 1). Additionally, some mice from the LT-CON group were switched from the LT-CON dietary program to the ST-CR dietary program (FIG. 1). This switching or crossover feeding further distinguished the 47 genes whose expression was altered by LT-CR. In one embodiment, the switched feeding fractionates or categorizes the 47 genes into 4 subgroups (discussed below) according to their response to changes in caloric intake as illustrated in FIG. 2A. The differences in the dynamics of changes in mRNA levels suggest that CR involves multiple complex molecular mechanisms in its effects on gene expression. Moreover, when these 47 genes were sorted according to the mode of regulation (positive or negative), the 4 subgroups were further separated into 7 gene clusters as illustrated in FIG. 2B. Genes assemble into clusters most likely because of similarities in the molecular mechanisms of their regulation. For example, several genes may have a common regulatory factor (e.g., enhancer sequences) or a common signal transduction pathway, and these common features are revealed through the gene clusters identified as a result of switching the diet programs. Thus, this switching allows for motif discovery.

FIGS. 2A-2B illustrate the effects of switched or crossover feeding on gene expression in heart tissue which was the source of the RNA in one exemplary embodiment. LT-CR altered the expression of 47 genes. The genomic effects of an 8-21 week switch of LT-CR and LT-CON mice to opposite diets further distinguished these 47 genes into 4 subgroups (FIG. 2A). A subgroup of 35 genes for which expression is altered by LT-CR but unaffected by either of the dietary regimen switches to the opposite diet, ST-CON or ST-CR dietary regimen. A subgroup of 8 genes for which ST-CR reproduced the gene expression changes induced by LT-CR. A subgroup of 1 gene for which ST-CON did not reverse the gene expression changes induced by LT-CR. Finally, a subgroup of 3 genes for which ST-CR reproduced but ST-CON did not reverse the gene expression changes induced by LT-CR.

The 47 genes were further sorted according to the direction of the changes in gene expression across the different experimental conditions. This sorting further segregated the 4 subgroups of genes into 7 gene clusters with similar patterns of expression (FIG. 2B). Cluster 1 (2 genes) illustrates that the increase in mRNA levels by LT-CR was reproduced by ST-CR but was not reversed by ST-CON treatment. Cluster 2 (1 gene) illustrates that the increase in mRNA levels by LT-CR was neither reproduced by ST-CR nor reversed by ST-CON treatment. Cluster 3 (1 gene) illustrates that the increase in mRNA levels by LT-CR was reproduced by ST-CR and was reversed by ST-CON treatment. Cluster 4 (21 genes) illustrates that the increase in mRNA levels by LT-CR was not reproduced by ST-CR but was reversed by ST-CON treatment. Cluster 5 (14 genes) illustrates that the decrease in mRNA levels by LT-CR was not reproduced by ST-CR but was reversed by ST-CON treatment. Cluster 6 (7 genes) illustrates that the decrease in mRNA levels by LT-CR was reproduced by ST-CR and was reversed by ST-CON treatment. Cluster 7 (1 gene) illustrates that the decrease in mRNA levels by LT-CR was reproduced by ST-CR but was not reversed by ST-CON treatment.

These genes, it is believed, congregated into clusters because of similarities in their expression profiles. Genes in the same cluster are thought to be regulated by similar mechanisms and thus, the regulatory sequences such as 5′ upstream regions of the genes can be analyzed to identify shared cis-regulatory elements. DNA sequence motifs specific to expression clusters constitute the primary hypothesis for the cis-regulatory elements though which co-regulation of the genes within a cluster is achieved. Algorithms such as AlignACE have been used to identify known and novel motifs based on gene expression data from microarray experiments. Thus, promoter comparison between genes within clusters and genes of different clusters can identify potential binding sites for known or novel factors that might control gene expression during CR.

The exemplary methods discussed allow for ways to categorize genes. As apparent from FIGS. 2A-2B, genes are fractionated into clusters (or groups) as certain genes are similarly affected by a particular CR dietary regimen. Genes in the same cluster are likely to be transcriptionally co-regulated and their promoter regions can be analyzed for the presence of shared sequence motifs. Motif discovery begins by identifying genes that are co-regulated under different conditions by CR. Genes which respond in the same way to given physiological conditions are grouped together. For example, as illustrated in FIG. 2B, genes which are responsive to ST-CR and LT-CR form 2 clusters (3, 8); genes which are responsive to LT-CR only form 2 clusters (22, 14); and ST-CON further subdivides genes into 7 clusters (2, 1, 1, 21, 14, 7, 1). The expression of different genes can be stimulated or inhibited by the same regulatory factors and signal transduction systems.

The most parsimonious explanation for the co-behavior of each of these clusters of genes is that they are co-regulated by the same signal transduction pathway. Gene regulation in eukaryotes mainly involves transcription factors binding to short DNA sequence motifs located upstream of the coding region of genes. Thus, the upstream sequences of a set of co-regulated genes can be analyzed for shared cis-regulatory motifs (short DNA sequences). These known or unknown DNA sequence motifs (regulatory motifs) common to gene clusters are putative binding sites for transcription factors. Algorithms such as AlignACE have been used to identify known and novel sequence motifs based on gene expression data from microarray experiments. Thus, promoter comparison within clusters and genes can identify potential binding sites for known or novel transcription factors that might control gene expression during CR. Knowledge of the identity of the transcription factors bound by the putative regulatory motifs will suggest which signal transduction systems may be responsible for the regulation of the genes by CR. The signal transduction systems responsible for gene regulation by many transcription factors are known. The signal transduction systems responsible for regulation of the activity of other transcription factors, including novel transcription factors which may be identified, may be determined experimentally. Drugs which alter the activity of identified, known signal transduction systems may be possible candidate CR mimetics. In other cases, potential CR mimetics which alter the activity of the identified signal transduction systems may be identified experimentally by monitoring some feature of the activity of the signal transduction system. This feature might be, for example, the phosphorylation or other modification of the structure or activity of a protein or changes in the activity of a specific gene. In this way, motif discovery may aid in the discovery or development of pharmaceuticals capable of mimicking the life- and health-span extending effects of CR.

Table 2 illustrates that LT-CR affects genes in the extracellular matrix (ECM) and cytoskeleton. LT-CR decreased the expression of several collagen encoding genes (e.g., procollagen genes U03419, X58251, and X52046). In the myocardium, a collagen matrix maintains the heart architecture, elasticity of the ventricles and vessels and the myocyte-capillary relationship. Previous studies in humans and rats show an increase in myocardial collagen associated with aging. See for example, Gazoti et. al., Age related changes of the collagen network of the human heart, Mech.Ageing Dev., 122: 1049-58, 2001 and Eghbali et. al., Collagen accumulation in heart ventricles as a function of growth and aging, Cardiovasc.Res., 23: 723-9, 1989. This increase of the myocardial collagen may contribute to the age-related decrease in ventricular and cardiovascular elasticity. Possible mechanisms for collagen accumulation include loss of myocytes which is a characteristic of the aging heart and age-related increase in systolic blood pressure. It has been shown through microarray studies of cardiomyopathies that increased expression of collagen and several other extracellular matrix proteins leads to fibrosis and impaired contractile function. Extracellular matrix, cytoskeleton, and their modification play important roles in cardiovascular functioning. As shown in Table 2, mice subjected to LT-CR showed decreased expression of collagen genes (e.g., U03419, X58251, and X52046). Additionally, mice subjected to ST-CR also showed decreased expression of collagen genes (e.g., U03419, X58251, X52046, and M15832). In contrast, mice under a control feeding program showed increased expression of collagen genes (e.g., U03419, X58251, and X52046) relative to mice in a CR dietary regimen. The decreased expression of extracellular matrix genes in CR (LT-CR or ST-CR) mice suggests less fibrosis and more elasticity in the myocardium of CR mice as opposed to the control mice. These effects may be part of the anti-aging strategy of CR to delay the age-25 associated decline in cardiovascular hemodynamics. The results indicate that mice subjected to CR may have extended longevity or delayed onset of age-related ventricular diseases since the expression of collagen genes are decreased as a result of CR.

Table 2 also illustrates that CR alters the expression of other extracellular matrix genes. For example, CR increased the expression of tissue inhibitor of metalloproteinase 3 gene which is a physiological inhibitor of matrix-degrading endopeptidases. Matrix remodeling results from a shift in the balance between metalloproteinases and their inhibitors. Disruption of this balance has been implicated in pathological states including cardiovascular diseases where tissue inhibitor of metalloproteinase activity was decreased. Thus, the results indicate that CR may delay the onset of cardiovascular diseases through decreasing tissue inhibitor of metalloproteinase activity. Additionally, CR decreased the expression of cysteine rich protein b1 gene. The product of this gene associates with extracellular matrix and binds directly to integrins to support cell adhesion and induces cell migration. Cysteine rich protein b1 expression is associated with the cardiovascular system during embryonic development. Later in life, its expression has been linked to angiogenesis and tumor growth.

Additionally, CR decreased the expression of microtubule-associated protein tau which promotes microtubule assembly and regulates cytoskeletal-membrane interactions. Tau is associated with Alzheimer's disease and was thought to be a neuron-specific protein. Tau is also expressed in the heart and other tissues. Even though the role of tau in cardiac microtubule assembly has not been shown yet, increased microtubule density is linked to contractile dysfunction in cardiac hypertrophy. Additionally, CR increased the expression of transgelin which plays a role in cytoskeleton organization and regulates smooth muscle cell morphology. Its expression is elevated in models of endothelial injury where transgelin is thought to mediate the conversion of myofibroblasts into smooth muscle cells. Moreover, transgelin is in human atherosclerotic plaque. These positive CR effects on the expression of EMC, cytoskeletal, signal transducer, and metabolism genes may be involved in retardation of cardiovascular diseases such as atherogenesis and hypertension.

Table 2 further illustrates that CR increased the expression of stearoyl-CoA desaturase gene, which is a rate-limiting enzyme in the synthesis of unsaturated fatty acids. The balance between saturated and monounsaturated fatty acids directly influences the membrane fluidity and its physical properties, and alterations in the ratio of these fatty acids have been implicated in many pathologies including vascular and heart diseases. Changes in lipid composition and decreased membrane fluidity occur with aging in several tissues. Thus, CR enhances membrane fluidity by increasing the desaturase gene expression.

Table 2 also illustrates that CR increases the expression of cytosolic acyl-CoA thioesterase 1 which controls levels of acyl-CoA/free fatty acids in the cytosol by hydrolysis of acyl-CoAs. While in tissues such as liver and kidneys thioesterases regulate gene transcription via nuclear receptors, cardiac thioesterases seem to be involved in the release of arachidonic acid (AA) from cellular phospholipids. AA can be metabolized to various cardioactive compounds, including prostanoids, leukotrienes, and epoxyeicosatrienioic acids. These metabolites and AA itself modulate a variety of systems in cardiomyocytes, including ion channels, gap junctions, and protein kinase C activity. More interestingly, the effects of AA on cardiac contractility combine a positive effect at low AA concentrations and a negative effect at high AA concentrations. The relative activation of the positive and negative pathways determines the nature of the final response. The effects of CR on cardiac cytosolic acyl-CoA thioesterase gene expression may be a fine tuning of these opposed pathways to result in an improved heart function.

Table 2 also illustrates that CR alters the expression of other metabolic genes. The expression of ADP-ribosyltransferase 3 gene, which is involved in posttranslational processing of nascent proteins, was increased by CR. The functional effects of the ADP-ribosyltransferase 3 gene differ depending on the tissue. In the skeletal muscle, the ADP-ribosyltransferase 3 gene ribosylates integrin to affect cell-cell and cell-matrix interactions. The role of ADP-ribosyltransferase 3 in cardiac muscle has not yet been determined. CR also increased the expression of the carbonic anhydrase 14 gene, which is most abundant in the kidney and heart. Carbonic anhydrase participates in various physiological processes including acid-base balance and ion transport. In the heart, acid-base homeostasis is important because of the pH sensitivity of myocardial contractility. Moreover, the failing myocardium is characterized by reduced carbonic anhydrase activity. The results here also indicate that CR delays progression toward cardiovascular diseases.

Table 2 further illustrates that CR alters the expression of several growth factor genes. CR decreased the expression of epithelial membrane protein I gene which has been implicated in tumorigenesis. CR increased the expression of p53 regulated PA26 nuclear protein gene which is a regulator of cellular growth and plays a role in tumor suppression. CR decreased the expression of the interferon induced transmembrane protein 3-like gene. It has been suggested that interferon-inducible transmembrane proteins transduce the antiproliferative activity of interferon. The implications of these opposed effects of CR on growth in the heart are unclear. In addition, beyond birth, cardiac growth occurs by hypertrophy rather than hyperplasia and primary tumors of the heart are rare.

Table 2 further illustrates that CR decreases the expression of several signal transducers relevant to cardiovascular diseases. CR decreases the expression of G protein-coupled receptor kinase 5 which is one of the two major G protein-coupled receptor kinases expressed in the heart. Increased expression and activity of these kinases have been shown to play an important role in the development of cardiac hypertrophy and congestive heart failure. Myocardial levels of G protein-coupled receptor kinase 5 mRNA and protein content are increased in experimental congestive heart failure. In addition, transgenic over expression of G protein-coupled receptor kinase 5 in mice leads to a significant decrease in myocardial performance. These results suggest that the CR-related decreased expression of this gene may improve and maintain healthy myocardial functioning. CR also decreased the expression of three other genes implicated in cardiovascular diseases, Ribosomal protein S6 kinase, 90 kD, polypeptide and stromal cell derived factor 1 and natriuretic peptide precursor type B. Ribosomal protein S6 kinase has been found to be activated in failing myocardium. Stromal cell derived factor 1 expression is induced in a permanent coronary artery occlusion model of myocardial infarction in rat. Ventricular expression of natriuretic peptide type B is increased in animal models of congestive heart failure. Increased production of this cardiac hormone is a marker of left ventricular dysfunction and has prognostic significance in patients with congestive heart failure. Since higher expression levels of natriuretic peptide type B are considered a protective response against myocardial damage, the lower expression levels in CR animals may reflect a healthier myocardium and thus, a more efficient cardiac function.

Table 2 further illustrates that CR affects genes associated with immune response and inflammation. Expression of genes related to inflammation, such as complement component 1, q subcomponent, c polypeptide and histocompatibility 2, k region locus 2 were decreased in CR mice. Cardiomyocytes and endothelial cells express MHC (major histocompatibility complex) class I and II antigens in and around inflammatory regions in the heart. Both MHC class II genes and the early genes of the classical complement system are expressed at low levels in resting macrophages and upregulated by activation of macrophages. Decreased expression of such genes suggests that CR may ameliorate inflammation in CR mice.

Table 2 further illustrates that CR affects genes associated with stress response and xenobiotic metabolism. CR increased the expression of cytochrome P450 enzyme 2 e1. This enzyme is expressed most highly in the liver where it metabolizes a broad spectrum of drugs and endogenous substances. However, it is also expressed in the heart. It is still not known if cytochrome P450 enzymes contribute significantly to drug and xenobiotic metabolism in the heart. CR also increased the expression of thioether S-methyltransferase which plays a role in the detoxification and solubilization of endogenous and exogenous sulfur- and selenium-containing compounds. Even though the physiological role of cytochrome P450 enzymes and thioether S-methyltransferase in the heart is still unclear, the increase of their expression by CR suggests they may play a role in protecting the heart against xenobiotics. However, the cytochrome P450 system was shown to modulate cardiomyocyte contraction in cell culture through metabolism of arachidonic acid. This suggests that cytochrome P450 enzymes, in the heart, may be involved in intracellular signal transduction

While particular embodiments of the present invention have been shown and described, it will be obvious to those skilled in the art that changes and modifications can be made without departing from this invention in its broader aspects and, therefore, the appended claims are to encompass within their scope all such changes and modifications as fall within the scope of this invention.

We claim:

1. A method of analyzing genes comprising:
administering a long term control (LT-CON) dietary program to a LT-CON group and a long term caloric restriction (LT-CR) dietary program to a LT-CR group for a first predetermined period, said LT-CON group and said LT-CR group comprised of similar mammalian samples;
after said first predetermined period, dividing said LT-CON group to a ST-CR group and a LT-CON continuation group, and switching said ST-CR group to a short-term caloric restriction (ST-CR) dietary program while maintaining said LT-CON continuation group on said LT-CON dietary program for a second predetermined period;
after said first predetermined period, dividing said LT-CR group to a ST-CON group and a LT-CR continuation group, and switching said ST-CON group to a short-term control (ST-CON) dietary program while maintaining said LT-CR continuation group on said LT-CR dietary program for said second predetermined period; and
comparing gene expression effects among said ST-CR group, said LT-CON continuation group, said ST-CON group, and said LT-CR continuation group.

2. The method of claim 1 wherein said comparing comprises comparing gene expression in said ST-CR group, ST-CON group, and LT-CR continuation group relative to said LT-CON continuation group.

3. The method of claim 1 further comprises fractionating genes into clusters based on how said genes are affected by switching dietary programs.

4. The method of claim 1 further comprising validating said gene expression effects using a method other than a microarray.

5. The method of claim 1 wherein said first predetermined period is about several months to about 36 months.

6. The method of claim 1 wherein said second predetermined period is about 1 day to about 8 weeks.

7. The method of claim 1 further comprising:
comparing gene expression effects between said LT-CR continuation group and said LT-CON continuation group;
comparing gene expression effects between said ST-CR group and said LT-CON continuation group; and
comparing gene expression effects between said ST-CON group and said LT-CON continuation group.

8. The method of claim 4 wherein said method other than a microarray is one of real time PCR, Northern blot, Western blot, primer extension, dot blot, and activity assays.

9. A method of identifying at least one regulatory nucleic acid sequence motif for a group of genes comprising:
administering a LT-CON dietary program to a LT-CON group and a LT-CR dietary program to a LT-CR group for a first predetermined period, said LT-CON group and said LT-CR group comprised of similar mammalian samples;
after said first predetermined period, dividing said LT-CON group to a ST-CR group and a LT-CON continuation group, and switching said ST-CR group to a ST-CR dietary program while maintaining said LT-CON continuation group on said LT-CON dietary program for a second predetermined period;
after said first predetermined period, dividing said LT-CR group to a ST-CON group and a LT-CR continuation group, and switching said ST-CON group to a ST-CON dietary program while maintaining said LT-CR continuation group on said LT-CR dietary program for said second predetermined period;
comparing gene expression effects among said ST-CR group, said LT-CON continuation group, said ST-CON group, and said LT-CR continuation group; and
identifying genes that exhibit similar behaviors for each of said ST-CR group, said LT-CON continuation group, said ST-CON group, and said LT-CR continuation group to identify genes affected by said switchings.

10. The method of claim 9 wherein said identifying comprises identifying at least one sequence, said at least one sequence is at least a portion of a regulatory sequence.

11. The method of claim 9 further comprises fractionating genes into clusters based on how said genes are affected by switching dietary programs.

12. The method of claim 9 wherein said mammalian samples includes mice.

13. The method of claim 9 wherein said first predetermined period is about several months to about 36 months.

14. The method of claim 9 wherein said second predetermined period is about 1 day to about 8 weeks.

15. The method of claim 9 further comprising:
comparing gene expression effects between said LT-CR continuation group and said LT-CON continuation group;
comparing gene expression effects between said ST-CR group and said LT-CON continuation group; and
comparing gene expression effects between said ST-CON group and said LT-CON continuation group.

16. The method of claim 9 further comprising:
validating said gene expression effects using a method other than a microarray.

17. The method of claim 16 wherein said method other than a microarray is one of real time PCR, Northern blot, Western blot, primer extension, dot blot, and activity assays.

18. A method of reducing collagen accumulation in mammals:
administering a CR dietary program to a mammalian group for a predetermined period.

19. The method of claim 18 wherein said CR dietary program includes a LT-CR dietary program and a ST-CR dietary program.

20. The method of claim 18 wherein said administering a CR dietary program further comprising:
administering a LT-CON dietary program to a LT-CON group and a LT-CR dietary program to a LT-CR group for a first predetermined period, said LT-CON group and said LT-CR group comprised of similar mammalian samples;
after said first predetermined period, dividing said LT-CON group to a ST-CR group and a LT-CON continuation group, and switching said ST-CR group to a ST-CR dietary program while maintaining said LT-CON continuation group on said LT-CON dietary program for a second predetermined period; and
after said first predetermined period, dividing said LT-CR group to a ST-CON group and a LT-CR continuation group, and switching said ST-CON group to a ST-CON dietary program while maintaining said LT-CR continuation group on said LT-CR dietary program for said second predetermined period.

21. The method of claim 20 wherein said first predetermined period is about several months to about 36 months.

22. The method of claim 20 wherein said second predetermined period is about 1 day to about 8 weeks.

23. The method of claim 20 wherein said mammalian samples includes mice.

24. A method of identifying a compound that potentially reduces collagen accumulation in at least one of heart or blood vessels:
obtaining control data from an administering of a feeding program to a first mammalian group;
administering an effective dosage of a test compound to a second mammalian group;
comparing at least one of collagen gene expression or collagen accumulation between said first mammalian group and said second mammalian group; and
identifying said chosen pharmaceutical agent to be potentially effective in reducing collagen accumulation based at least in part on said comparing.

25. The method of claim 24 wherein said feeding program includes a CR dietary program.

26. The method of claim 25 wherein CR dietary includes at least one of a LT-CR dietary program and a ST-CR dietary program;

27. The method of claim 24 wherein said control data results from comparison of gene expression levels in CR relative to a control.

28. The method of claim 24 wherein gene expression in said second mammalian group reproduces gene expression in said first group.

29. The method of claim 24 wherein said first and second mammalian groups include mice.

30. The method of claim 24:
administering a LT-CON dietary program to a LT-CON group and a LT-CR dietary program to a LT-CR group for a first predetermined period, said LT-CON group and said LT-CR group comprised of similar mammalian samples;
after said first predetermined period, dividing said LT-CON group to a ST-CR group and a LT-CON continuation group, and switching said ST-CR group to a ST-CR dietary program while maintaining said LT-CON continuation group on said LT-CON dietary program for a second predetermined period; and
after said first predetermined period, dividing said LT-CR group to a ST-CON group and a LT-CR continuation group, and switching said ST-CON group to a ST-CON dietary program while maintaining said LT-CR continuation group on said LT-CR dietary program for said second predetermined period;
wherein said administering an effective dosage of a chosen pharmaceutical agent is for said second predetermined period.

31. The method of claim 24 wherein said first predetermined period is substantially longer than said second predetermined period.

32. The method of claim 24 wherein said first predetermined period is about several months to about 36 months.

33. The method of claim 24 wherein said second predetermined period is about 1 day to about 8 weeks.

34. A method of identifying a compound that potentially reduces collagen accumulation in at least one of heart and blood vessels comprising:
obtaining control data from an administering of a CR dietary program to one sample group;
administering a dosage of a compound to another sample group;
comparing at least one of collagen measurement resulting from said CR dietary program to at least one collagen measurement resulting from said administering a dosage of a compound; and
identifying said compound to be potentially effective in reducing collagen accumulation based at least in part on said comparing.

35. A method of analyzing genes comprising:
administering a first type of CR dietary program for a first period of time for a first sample;
administering a second dietary program for the first sample after the first period of time;
administering a control diet to a second sample; and
analyzing gene expression effects between the first sample and the second sample.

36. The method of claim 35 wherein said first type of CR dietary program is one of a LT-CR dietary program and a ST-CR dietary program.

37. The method of claim 35 wherein said second dietary program is one of a LT-CR dietary program and a ST-CR dietary program.

38. The method of claim 35 wherein said first type of CR dietary program is one of a LT-CON dietary program and a ST-CON dietary program.

39. The method of claim 35 wherein said first type of CR dietary program is one of a LT-CON dietary program and a ST-CON dietary program.

40. The method of claim 35 wherein said analyzing comprises categorizing genes into groups based on increases and decreases in mRNA levels in the first and the second samples.

41. The method of claim 35 wherein said first sample and said second sample include mice.

42. The method of claim 35 wherein said first period of time is about several months to about 36 months.

43. The method of claim 35 wherein said second period of time is 2 months.

44. A method for identifying targets for interventions comprising:
comparing gene expression levels or protein activity levels in a sample exposed to a first type of CR and to a second type of CR; and
identifying genes that appear to have similarity in both the first and the second types of CR.

45. The method of claim 44 wherein said first type of CR dietary program is one of a LT-CR dietary program and a ST-CR dietary program.

46. The method of claim 44 wherein said second dietary program is one of one of a LT-CR dietary program and a ST-CR dietary program.

47. The method of claim 44 wherein said analyzing comprises categorizing genes into clusters based on increases and decreases in mRNA levels in the first and the second samples.


SEQUENCE LISTING (10 sequences)

SEQ ID NO: 1 - DNA, 50 bp, Mus musculus
ctgggtcaagtcaccctgtgaagaccgatgaaacagacaccagtctcaag

SEQ ID NO: 2 - DNA, 50 bp, Mus musculus
ccaacaagcatgtctggttaggagatgttctgagaagcacggttggctag

SEQ ID NO: 3 - DNA, 50 bp, Mus musculus
ccctggtatcattgtacccaccttggatgggactcaactgcatcgggtag

SEQ ID NO: 4 - DNA, 50 bp, Mus musculus
tgctgggtaggtaggtgctctaatcgatacatgtgggaacattgcaggac

SEQ ID NO: 5 - DNA, 50 bp, Mus musculus
agaagtctctgaagctgatgggatcgccttgcgtgtttgatattcaaaga

SEQ ID NO: 6 - DNA, 50 bp, Mus musculus
aattgtatcgcgaacgcagaatataaaggttgttcctaccagagtcttca

SEQ ID NO: 7 - DNA, 50 bp, Mus musculus
tctgagccccttgtacagaactacagacccagcatctctcctgtggtata

SEQ ID NO: 8 - DNA, 50 bp, Mus musculus
tcttagccctgacagctctgaggtgacttctccctgcttactccaggatg

SEQ ID NO: 9 - DNA, 50 bp, Mus musculus
agctcttgaaggaccaaggcctcactatcttgtgcccaaagcagcttgag

SEQ ID NO: 10 - DNA, 50 bp, Mus musculus
ccagctgaaatgtaggctgtagcaaacaggagtctgaacacaggcagaag